(19) United States
(12) Patent Application Publication
(10) Pub. No.: US 2020/0316320 A1
(43) Pub. Date: Oct. 8, 2020

(54) LAPAROSCOPIC INSUFFLATION DEVICES AND RELATED METHODS

(71) Applicant: InsuffleX, LLC, Salt Lake City, UT (US)

(72) Inventors: Andrew J. Nelson, Layton, UT (US); Adam Cortland Kessel, Salt Lake City, UT (US)

(21) Appl. No.: 16/843,683

(22) Filed: Apr. 8, 2020

Related U.S. Application Data
(60) Provisional application No. 62/830,717, filed on Apr. 8, 2019.

(51) Int. Cl. A61M 13/00

(52) U.S. Cl. CPC A61M 13/003; A61M 2205/75; A61B 1/3132; A61M 2205/8206; A61M 2205/3337

(57) ABSTRACT

Insufflators and related insufflation methods. In some embodiments, an insufflator assembly may comprise a first port fluidly coupled with and configured to provide access to ambient air, a gas pump fluidly coupled with the first port, and a second port configured to deliver pressurized ambient air generated from the gas pump for delivery to a patient. The assembly may further comprise a battery configured to provide a portable energy source for delivery of electrical energy to the gas pump.

### CROSS-REFERENCE TO RELATED APPLICATIONS

This application claims the benefit under 35 U.S.C. § 119(e) of U.S. Provisional Patent Application No. 62/830,717, which was filed Apr. 8, 2019 and titled “DEVICE FOR INSUFFLATION DURING LAPAROSCOPIC PROCEDURES,” which is hereby incorporated herein by reference in its entirety.

### BACKGROUND

The present disclosure relates to insufflation devices. More specifically, in some embodiments, the device may utilize electric air pumps, battery or AC/DC power, have one or more filters, and is preferably portable.

Laparoscopic surgery, also called minimally invasive surgery, is used for a variety of medical purposes, such as evaluating cancer or removing a lymph node. In laparoscopic surgery, the surgeon makes a small incision, typically about 0.5 inches long, and the laparoscope, a thin instrument that has a camera attached, is inserted through the small incision. The laparoscope allows for the surgeon to view a particular area of the body, such as organs in the abdomen area. If a problem is detected, other surgical instruments can be used, and the problem can be resolved by utilizing the small incision, avoiding open surgery, where the surgeon has to make a large cut through the skin.

To perform a laparoscopic surgery, the patient has to be under anesthesia, after which a small incision is made, typically near the navel or in the abdomen area. The laparoscope is then inserted through the small incision. The abdomen is filled with gas, usually carbon-dioxide, so that the organs and body cavities can be seen more clearly. Currently, the devices that fill the abdomen with gas typically require both reliable access to bottled carbon dioxide gas, and power through an outlet. Therefore, laparoscopic surgery is not easily performed globally, as safe sources of carbon dioxide are not always available and/or reliable access to central power sources is not guaranteed. This is especially true in third-world countries, conflict zones, and disaster/recovery areas. This presents great risks to patients because, rather than laparoscopic surgery, some patients may have to undergo open surgery, having larger wounds and a longer recovery time. Larger wounds also present an increased risk of infection. Thus, there is a need to make laparoscopic surgery more globally accessible.

### SUMMARY

The summary of this device is not intended to describe each illustrated embodiment or every possible implementation of the device. The figures and the detailed description that follow, however, do particularly exemplify embodiments of the device. In preferred embodiments, the invention relates to a device for insufflation during laparoscopic surgery. In some embodiments, the device may comprise electric air pumps, powered by either battery or AC/DC power, to supply gas to the patient during laparoscopic surgery.

Additional features and advantages of the device will be set forth in the description that follows, and in part will be obvious from the description, or may be learned by the practice of the invention. The features and advantages of the device may be realized and obtained by means of the instruments and combinations particularly pointed out in the appended claims. These and other features of the device will become more fully apparent from the following description and appended claims or may be learned by the practice of the invention as set forth hereinafter.

In a more specific example of a insufflator assembly, such as a portable insufflator assembly, the assembly may comprise a first port fluidly coupled with and configured to provide access to ambient air; a gas pump fluidly coupled with the first port; and a second port configured to deliver pressurized ambient air generated from the gas pump for delivery to a patient. The assembly may further comprise a battery configured to provide a portable energy source for delivery of electrical energy to the gas pump.

Some embodiments may further comprise a filter configured to filter the ambient air prior to exiting from the second port. In some such embodiments, the filter may be part of a filter assembly, which filter assembly may be removable and/or disposable.

Some embodiments may further comprise one or more valves, such as a solenoid valve configured to open a venting port upon sensing a threshold pressure by the pressure sensor.

Some embodiments may further comprise a housing configured to enclose the gas pump and the battery. In some such embodiments, the housing may comprise a nylon material and may be formed using an additive manufacturing technique.

In some embodiments, the assembly may be configured to alternatively, or as a supplement to ambient air, deliver another gas, such as carbon dioxide. Thus, some such embodiments may comprise an adapter configured to couple with a carbon dioxide cannister to allow for alternatively delivering carbon dioxide through a delivery port, such as the same delivery port (the second delivery port) or a different delivery port.

Some embodiments may further comprise means for stepping down gas pressure from a carbon dioxide gas cannister to allow for, as an alternative to ambient air, delivery of carbon dioxide through a set of tubes through which the ambient air is delivered when the carbon dioxide cannister is disconnected from the medical insufflator assembly. This step-down in pressure may be achieved via a timed activation of a supply valve in some embodiments. For example, pressure may be recorded at a supply pressure sensor and compared to a simultaneously recorded pressure at a patient pressure sensor. This comparison between supply and operation pressures allows calculation of an appropriate valve activation time. Depending on the application, this process may occur between, for example, 10 and 100 times per second, which allows the device to maintain appropriate operating pressure for a given supply pressure.

In an example of a medical insufflator assembly according to some embodiments, the assembly may comprise an access port fluidly coupled with and configured to provide access to ambient air. A gas pump may be fluidly coupled with the access port. The assembly may further comprise a delivery port configured to deliver pressurized ambient air generated from the gas pump and a filter assembly configured to filter ambient air received from the access port. The filter assembly may be removable, replaceable, and/or disposable in some embodiments. The assembly may further comprise one or more sensors, such as a pressure sensor configured to sense an internal gas pressure. The assembly may further comprise one or more valves, such as a valve configured to open a venting port upon sensing a threshold pressure by the pressure sensor and/or a valve configured to supply air or another gas to the patient upon actuation.

In some embodiments, the insufflator assembly may be portable and may therefore comprise a battery configured to provide a portable energy source for delivery of electrical energy to the gas pump and/or other components of the assembly.

Some embodiments may further comprise an alternating current converter to allow for use of alternating current for charging the battery or operating the medical insufflator assembly using an alternating current power source.

Some embodiments may further comprise an alarm, such as a visual, audible, and/or tactile alarm configured to notify a user of a detection of a condition dangerous to a patient, such as a condition comprising at least one of a gas pressure and a gas flow rate exceeding a threshold.

In an example of a method for insufflating a patient during a surgical procedure, the method may comprise drawing ambient air into an insufflation device, such as in some implementations a portable insufflation device configured to operate using a direct current source, such as a battery. The ambient air may be pressurized within the insufflation device and then delivered through an incision in a patient using the insufflation device.

Some implementations may further comprise filtering the ambient air through a filter assembly; removing the filter assembly; and replacing the filter assembly with a new filter assembly. The filter assembly and/or other elements of the insufflation device, such as a housing, lid, and/or tubing, may be manufactured using an additive manufacturing technique.

Some implementations may further comprise coupling a carbon dioxide gas cannister with the portable insufflation device and delivering carbon dioxide gas through an incision in a patient using the portable insufflation device. The carbon dioxide gas may be delivered alone or as a supplement to ambient air.

Some implementations may further comprise sensing a gas pressure exceeding a threshold gas pressure and/or a gas flow rate exceeding a threshold gas flow rate. In response to sensing the gas pressure exceeding the threshold gas pressure and/or the gas flow rate exceeding the threshold gas flow rate, one or more valves may be actuated to prevent gas exceeding the threshold gas pressure from being delivered into the patient.

Some implementations may further comprise stepping down gas pressure from the carbon dioxide gas cannister from a first pressure at which the carbon dioxide cannister stores the carbon dioxide gas to a second, lower pressure to allow for, as an alternative to ambient air, delivery of carbon dioxide through a set of tubes through which the ambient air is delivered when the carbon dioxide cannister is disconnected from the portable insufflator device.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a schematic view of an insufflator according to some embodiments;

FIG. 2 is a schematic view of an insufflator according to alternative embodiments;

FIG. 3 is an exploded, perspective view of an insufflator;

FIGS. 4A and 4B are perspective views of a housing for an insufflator according to some embodiments; and

FIGS. 5A and 5B are perspective views of a filter assembly for an insufflator according to some embodiments.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

Currently, laparoscopic surgery, as described above, is not globally accessible because insufflators require outlet power and not all medical locations, particularly third-world countries, have outlet power. Thus, patients without access to laparoscopic surgery have to undergo open procedures where the surgeon cuts through the skin, and recovery time is greater than when laparoscopic surgery is done.

To make laparoscopic surgery globally accessible, in some embodiments, an insufflator device may be provided that is battery powered. Thus, such insufflator devices may be portable and used more widely in locations and places where outlet power may not be readily available.

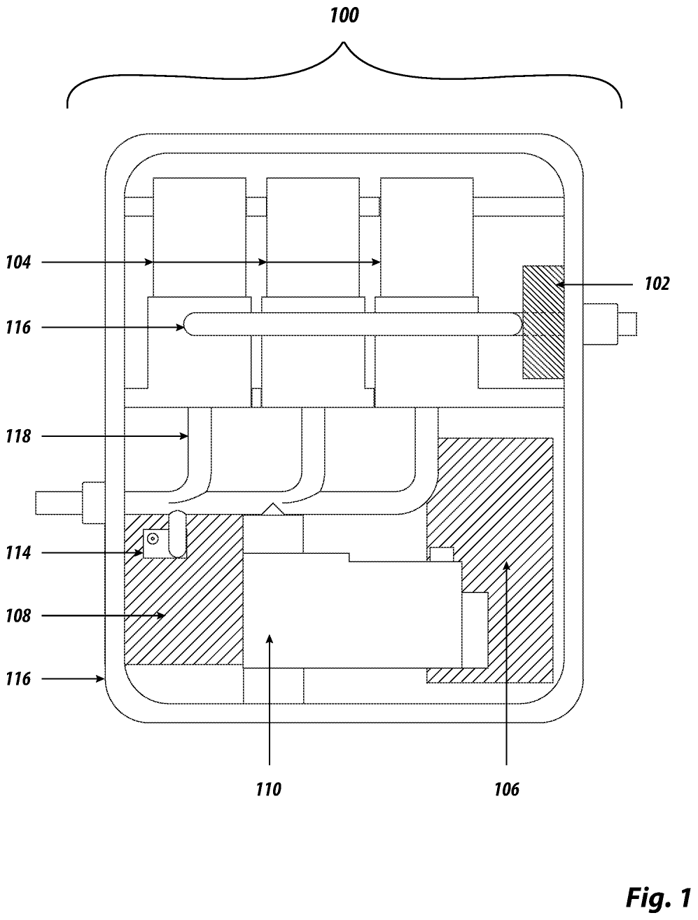

FIG. 1 illustrates an example embodiment of an insufflator device (the “device”). The device 100 includes an opening 116 to take air in. The opening 116 may be attached to a carbon-dioxide tank in some embodiments. The device has one or more filters. In this example embodiment, there is one large filter 102, although multiple filters may be used in other contemplated embodiments. In some embodiments, though, there may be dual filtration of air.

The device 100 may further comprise one or more air pumps 104. In this example embodiment, there are three air pumps 104, but in other embodiments there may be fewer or more air pumps. The device 100 further comprises a battery 106. For example, some embodiments may comprise a lithium-ion battery pack, which in some such embodiments may be removable to allow for charging and/or replacement as needed. In some embodiments, the device 100 may have more than one battery.

The device 100 may also include an outlet for DC and/or AC power. The device may also include a safety valve 110. Safety valve 110 may be configured to actuate when there are unsafe air pressure levels for the patient. For example, in some embodiments, safety valve 110 may be configured to actuate upon detecting a threshold pressure within the device 100, such as a pressure greater than about 45 mmHg, in some such embodiments and implementations for a time period longer than a predetermined interval of time, such as 5 seconds. In some embodiments, the safety valve 110 may be actuated upon sensing of a pressure exceeding a user-set pressure by a threshold amount, such as exceeding the pressure by more than 5 mmHg for a predetermined interval of time, such as 5 seconds. The safety valve 110 preferably vents gas upon detecting such a pressure threshold to prevent the gas from going to the patient. In some embodiments, the device 100 may include more than one valve that respond to gas pressure levels.

The device 100 also includes a circuit board 108. In some embodiments, the device 100 may include a user interface, such as a touchscreen or the like, to allow a user to operate the device 100. The device 100 may also include one or more pressure sensors 114, which may be communicatively coupled with the circuit board 108 and/or a control board to allow for transfer of data obtained by the sensor(s) 114. The device 100 further includes a tube 118 to provide the gas to the patient. The tube 118 may contain a filter if desired.

In some embodiments, the device 100 may include other electrical components, such as a voltage smoothing filter. Some embodiments may also include a flow rate sensor and/or an alarm that alerts the user to unsafe pressure levels.

The following description is given to illustrate how a physician or operator of some embodiments of the device 100 may use the device 100 in a specific method/application. This description is meant to be illustrative of the kinds of use to which the device 100 can be utilized and should not be interpreted as limiting the scope of the claimed device or its application. The following description relates to using the device 100 to guide tissue incision and/or closure and is just one application of the many applications for which the device 100 can be used.

A surgeon when performing laparoscopic surgery may use the device 100 to put carbon dioxide or filtered ambient air into a patient. Typically, the device 100 would specifically be used after the surgeon makes a small incision in the patient near the operating site. The device 100 may be used as long as the surgeon desires.

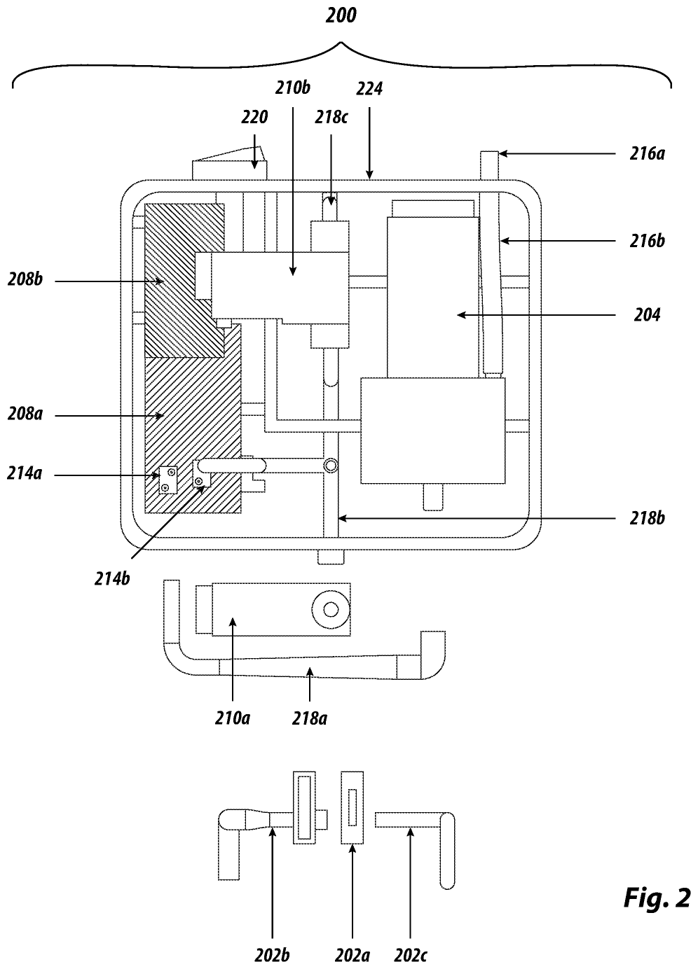

FIG. 2 is a schematic view of another example of an insufflation device 200 according to other embodiments. Device 200 may comprise a housing 224, which, in preferred embodiments, may comprise a 3D-printed housing that may be custom designed with integrated support for desired device components. In some embodiments, housing 224 may be manufactured using acrylonitrile butadiene styrene (ABS) or polyethylene terephthalate glycol (PETG). However, it is contemplated that in other embodiments housing 224 may comprise another suitable material, such as aluminum or the like.

Device 200 may further comprise a filter assembly, which preferably comprises a disposable/replaceable filter assembly. This filter assembly may be also be designed to be 3D printed as a single piece. The filter assembly can then be removed and cleaned or replaced by the user. In the depicted embodiment, the filter assembly may comprise a cartridge 202a, which may comprise a slot and/or chamber into which a filter media may be positioned. The cartridge 202a may fit within a corresponding slot of a filter housing 202b.

Distribution Tubes 202b and 202c may be provided to allow for coupling of the filter assembly to tubing of device 200. For example, distribution tube 202b may be fluidly coupled with the intake side of the device 200 and distribution tube 202c may distribute air or another gas from the filter to the patient and/or patient supply valve 210a.

Device 200 may further comprise one or more gas pumps 204. In some embodiments, pump 204 may comprise a micro air vacuum pump, preferably capable of delivering up to 15 L/minute.

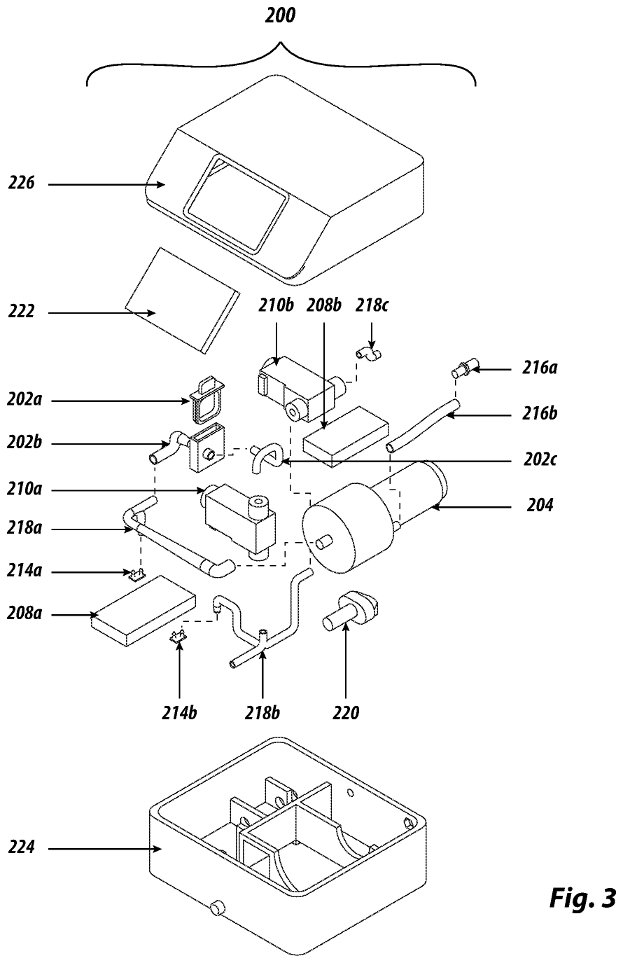

A microcontroller 208a may also be provided, which may be configured to receive input from the user via, for example, a touchscreen 222 (see FIG. 3). Microcontroller 208a may also be configured to receive sensor data, such as input from one or more pressure sensors. Microcontroller 208a may therefore control air flow rate, valve action, and/or display/IO functions. Microcontroller 208a may, in some embodiments, also record trends in pressure and air flow rate throughout a procedure, giving feedback to care providers regarding the patient's response to insufflation. Thus, some embodiments may further comprise a memory element, such as random-access memory (RAM) and/or a non-transitory computer-readable storage medium.

Device 200 may further comprise a power regulator 208b, which may comprise, for example, a “buck”-type power regulator. Regulator 208b may comprise a circuit board that accepts a 12V power source and outputs 5V. This may allow for a differential in voltage to different elements, such as a supply of 12V to pump 204 and/or valve 210 and 5V to the electronics and/or sensors. In some contemplated embodiments, however, power regulator 208b may be integrated into a main control board.

Device 200 may further comprise one or more valves, such as solenoid valves. For example, device 200 comprises a valve 210a that may be biased towards a closed position and actuated magnetically via voltage input. Thus, when there is no power to valve 210a, the valve 210a is closed. Valve 210a may be configured to open continuously upon detection of a threshold range of pressures by a pressure sensor, such as a pressure between about 1 mmHg and about 30 mmHg. Additionally, valve 210a may be configured to open periodically upon detection of a different threshold range of pressures by the same pressure sensor, such as a pressure between about 30 mmHg and about 3000 mmHg.

One valve may be used to control airflow from the filter housing to the patient, and another valve may control venting in case of emergency overpressure. Thus, a vent valve 210b may also be provided. In some embodiments, device 200 may only supply air or another gas to the patient when the patient-side valve is powered. In addition, preferably each of the valves is configured to actuate quickly, which is typically the case for solenoid valves, thereby allowing fast response in case of an over-pressure event or a user requested venting.

As alluded to above, preferably one or more sensors are used to detect and, ultimately, control, the pressure and/or airflow provided by device 200. Thus, device 200 comprises an ambient air supply sensor 214a and a patient sensor 214b.

Sensor 214a may be a “comparative” pressure sensor that, in some embodiments, may be rated for 0-1 PSI. Sensor 214a may be configured to operate by comparing the pressure from one input port to that of the other, and outputs a voltage relating to this proportion. This configuration allows the sensor to be factory calibrated.

An intake tube 216b may be coupled with pump 204, which intake tube 216b may be coupled with a DISS CO2 connector 216a. Thus, device 200 may be configured to selectively pump either ambient gas or CO2. Connector 216a may be configured to accept input from a central CO2 system or standalone CO2 tanks.

Various distribution tubes/tubing may also be provided as needed. For example, a distribution tube 218a may be configured to distribute air or another gas from the pump 204 to one or more pressure sensors and/or filters. Another distribution tube 218b may be configured to distribute air or another gas from the patient supply valve 210a to the patient pressure sensor 214b, the vent valve 210b, and the external insufflation tubing.

Preferably, all of the tubing provided in device 200 is, like housing 224, manufactured using 3D printing, preferably using a nylon filament. Such tubing also preferably comprises smooth size transitions and curves to facilitate laminar airflow. The thickness of the walls defining the tubing may, in some preferred embodiments, be less than or equal to about 2 mm, which may be beneficial to ensure an air-tight print. Preferably, the 3D printer settings should indicate 100% infill.

Device 200 may further comprise a vent tube 218c, which may extend to a peripheral edge of the housing 224 and may bring air or another gas from the vent valve 210b to the outside of the device 200.

A power switch 220, such as a rocker power switch, may also be provided, as shown in the exploded view of FIG. 3. In some embodiments, this switch may be a standardized power switch, which may be rated for 12V at up to 5A.

One or more user interface/user input elements may also be provided to allow a user to view and adjust settings, control the device, receive pressure readings, warnings, and the like to monitor a patient during a procedure. Thus, device 200 comprises a touchscreen 222. It is contemplated that touchscreen 222 may be replaced by any other suitable user interface elements, such as buttons, monitors, switches, dials, and the like, in alternative embodiments. An opening may be built into a case lid 226 to allow for acceptance of touchscreen 222. As with housing 224, lid 226 is preferable manufactured via 3D printing.

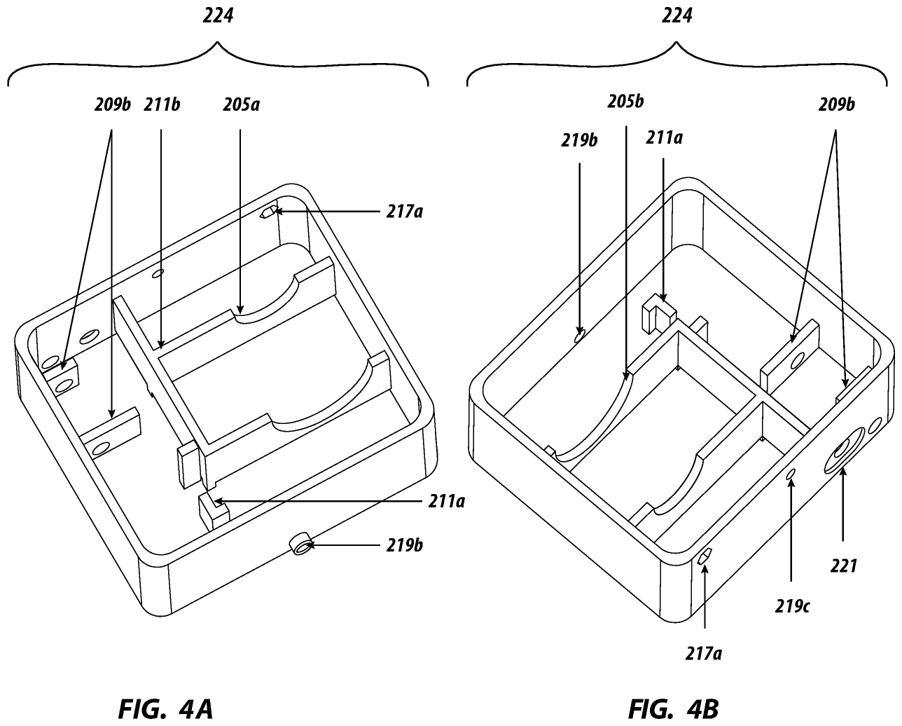

FIGS. 4A and 4B are perspective views of the housing 224, which illustrate in greater detail various features of this element of device 200. Thus, as shown in these figures, housing 224 comprises an integrated support structure 225 having a plurality of walls, openings, chambers, and the like, for keeping the various components of device 200 in place. Included in support structure 225 are semi-circular cutouts 205a/205b. These support walls and cutouts 205a/205b of support structure 225 may be configured to engage and secure pump 204 in its proper position. Support structure 25 may also comprise integrated supports 209b, which may be configured to hold power regulation board 208b in place. Similar integrated supports 211a/211b may be provided to hold valves 210a and 210b in place.

Various openings/holes are provided throughout housing 224 and/or support structure 225 to facilitate air flow and wiring, some of which are specifically discussed below. For example, an opening 219c may be provided to allow air or another gas to vent to the atmosphere via internal tube 218c. Another opening 217a may be provided and fitted for air intake connector 216a. Similarly, housing 224 may comprise an opening 219b for air/gas outlet, which connects internal tube 218b to external patient tubing (not shown). As shown in FIG. 4B, an inset 221 may be provided for receiving the external power switch 220.

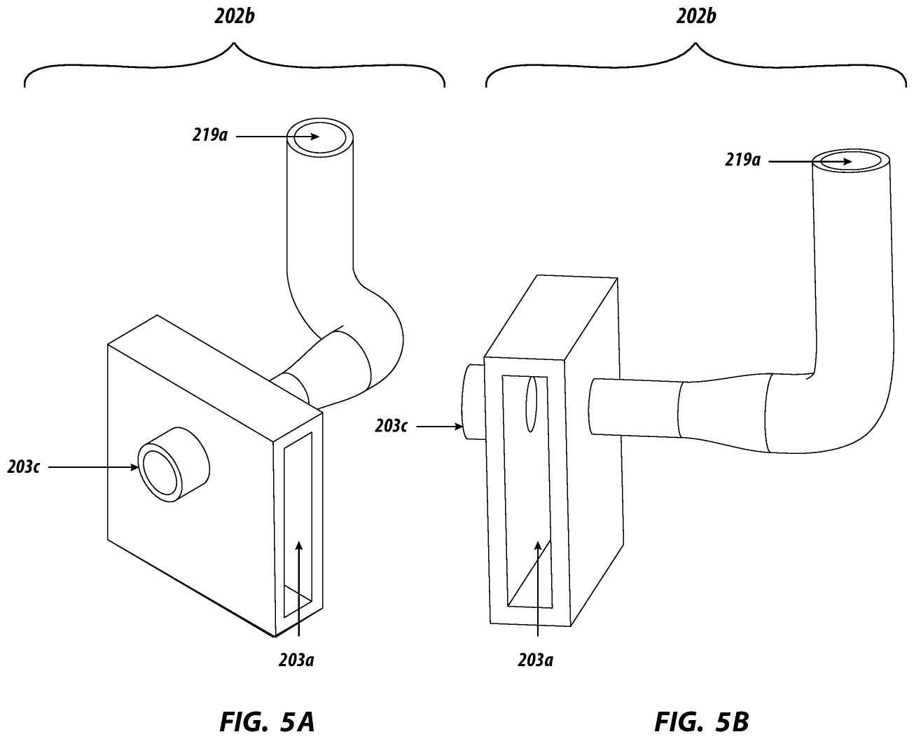

FIGS. 5A and 5B are perspective views of a disposable and/or replaceable filter housing 202b. As shown in these figures, filter housing 202b comprises a slot or chamber 203a, which is configured to receive a filter cartridge 202a containing filter media. Preferably, filter housing 202b, like many of the other elements of device 200, is manufactured using 3D printing technology.

Integral tubing 219a may extend from the portion of filter housing 202b defining chamber 203a and may comprise a joint to allow for connecting the filter housing 202b to tube 218a. On the opposite side of the portion of filter housing 202b defining chamber 203a, another piece of tubing 203c may extend for connecting the filter housing 202b to tube 202c.

During operation, preferred embodiments of the invention, such as devices 100 or 200, may be configured to run in two separate modes: CO2 mode or ambient mode. Selection of CO2 mode may allow for the use of CO2, either as a supplement to ambient air or as an alternative to ambient air, for inflation and is regulated by the supply valve 210a. By contrast, ambient mode may allow for the use of filtered room air for inflation and is regulated by the micro air vacuum pump 204.

As mentioned above, the device control system may comprise two valves, namely, a supply valve 210a and a safety vent valve 210b. The device control system may further comprise two sensors, namely, a supply sensor 214a and a patient sensor 214b, both of which may be communicatively coupled with a microcontroller 208a. Both valves are preferably closed when the device is powered off and may be cycled on device start to ensure proper function. Preferably, if any valve or sensor fails to initialize correctly during calibration, the device will not continue to set-up, and will display an appropriate error message and/or provide another warning signal, audible or visible.

As previously mentioned, the device may be configured to accept ambient air or CO2 through an industry standard CO2 connector 216a. An intake tube 216b carries air from the input connector to the pump 204. From the pump 204, air and/or gas is carried through a distribution tube 218a to the supply sensor and the filter housing 202b. After filtration, air/gas is carried via another distribution tube 202c to the supply valve. From the supply valve, air/gas is carried via a central patient supply tube 218b to the patient pressure sensor, the external insufflation port, and the vent valve 210b. From the vent valve 210b, air/gas can be removed from the device or patient via the vent tube 218c and an external vent port. This configuration allows the supply valve and vent valve to operate independently, ensuring accurate pressure regulation and proper fail-safe operation.

When powered on, preferably the device will initially run a calibration phase. During calibration, valves remain closed and the supply sensor will take a reading to determine if there is any back pressure in the system. In the setting of CO2 supplementation, the device may be configured to pressurize the tubing up to the first valve without running the pump.

Because standard medical CO2 tanks can be filled to 2000 PSI or greater, a pressure regulator is typically connected between the CO2 tank and the insufflator, limiting supply pressure to no more than 50 PSI. In some hospitals, a central CO2 system may be in place, which is typically limited to 50 PSI or less. Thus, in preferred embodiments, if the device supply sensor 214a senses a pressure within a designated range, which may between about 1 PSI and about 60 PSI in some embodiments, on the supply side, the device may be configured to calibrate to CO2 mode and initiate CO2 insufflation and/or CO2 supplementation along with ambient air. Similarly, if the device supply sensor senses less than a threshold pressure, the device may be configured to calibrate to ambient mode by initiating pressure regulation via the pump.

In some embodiments, supply pressures above a threshold pressure, such as about 60 PSI for example, may risk device failure. Thus, upon detection of a supply pressure above a threshold, such as about 50 PSI in some embodiments, the device may be configured to issue a supply pressure warning. In some such embodiments, calibration may not complete until supply pressure falls below the threshold pressure for a predetermined period of time, such as at least 30 continuous seconds in some embodiments. Preferably, the device is configured such that excessive pressures will cause internal tubing joints to fail first such that excess air/gas will be vented through a vent port of the device. In any event, preferably the device is configured to prevent runaway/excessive supply pressures to be delivered to the patient under any circumstances.

In either ambient air or CO2 mode, in some embodiments the device may be configured to enter a set-up phase, in which it accepts a desired operating pressure as input from a user. The device may also prompt users to review other device settings, such as vent control, data collection and storage, etc. The device may then be configured to maintain the desired input pressure within a margin for error, such as about plus or minus 1 mmHg for example, throughout the duration of the operation.

Preferably, the device may be configured to maintain pressure between about 1 and about 30 mmHg (per user settings) and to avoid exceeding a threshold pressure, such as about 45 mmHg in some embodiments, for greater than a predetermined interval of time, such as about 15 seconds, cumulatively throughout the procedure. The device may also be configured to avoid exceeding a set pressure by a threshold increase in pressure, such as about 5 mmHg in some embodiments, for greater than a predetermined period of time, such as 5 seconds, per over-pressure event.

Pressure regulation in the device is preferably maintained through software. Throughout operation in CO2 mode, a patient sensor 214b (reading intra-abdominal pressure) may be compared to the supply sensor 214a (reading supply pressure) to provide feedback for regulation of the supply valve. In CO2 mode, the device may be configured to automatically actuate the supply valve and may add only enough ambient air to maintain desired operating pressure.

In ambient mode, the supply valve may be configured to stay open and the sensors should give approximately the same reading. During standard ambient operation, the pump speed may be controlled via software to precisely maintain desired operating pressure. In some circumstances, the device may be set to supplement ambient air pressure using the pump. In this use case, the pump may be configured to maintain a supply pressure within a predetermined range, such as between about 30 PSI and about 50 PSI, and operating pressure would be regulated by actuation of the supply valve, rather than via pump action directly, similar to operation in CO2 mode.

In either mode, preferably a safety vent valve 210b is in place to prevent over-inflation. If the patient sensor detects a pressure a threshold value, such as about 5 mmHg above the operating pressure, for a predetermined interval of time, such as more than 5 seconds, the supply valve 210a may be configured to automatically close and the pump automatically stop (if active). If pressure climbs further, the safety vent valve 210b may be configured to open and bring the pressure back down. Additionally, preferably both valves have the option of manual control, which may allow a user to add or remove a specific amount of air from the patient. To do this, a user may enter the device settings, enable manual control, select a desired bolus and/or vent pressure, and select a duration for which to hold adjusted pressure. With manual control enabled, the device may be configured to display selectable options, such as pressable buttons on a touchscreen display, which, when pressed, will automatically increase or decrease intrathoracic pressure by the set amount (between 1 and 10 mmHg, for example), hold pressure at this new value for the specified time period (between 1 and 20 seconds, for example), and then return the patient to an initial and/or standard operating pressure.

Examples of “means for stepping down gas pressure from a carbon dioxide gas cannister disclosed herein include sensors 214a and 214b, along with supply valve 210a.

It will be understood by those having skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. The scope of the present invention should, therefore, be determined only by the following claims.

1. A portable insufflator assembly, comprising:
a first port fluidly coupled with and configured to provide access to ambient air;
a gas pump fluidly coupled with the first port;
a second port configured to deliver pressurized ambient air generated from the gas pump for delivery to a patient; and
a battery configured to provide a portable energy source for delivery of electrical energy to the gas pump.

2. The portable insufflator assembly of claim 1, further comprising a filter configured to filter the ambient air prior to exiting from the second port.

3. The portable insufflator assembly of claim 1, further comprising a valve configured to open a venting port upon sensing a threshold pressure by the pressure sensor.

4. The portable insufflator assembly of claim 3, wherein the valve comprises a solenoid valve.

5. The portable insufflator assembly of claim 1, wherein the battery is removable.

6. The portable insufflator assembly of claim 1, further comprising a housing configured to enclose the gas pump and the battery, wherein the housing comprises a nylon material formed using an additive manufacturing technique.

7. The portable insufflator assembly of claim 1, further comprising an adapter configured to couple with a carbon dioxide cannister to allow for alternatively delivering carbon dioxide through a delivery port.

8. The portable insufflator assembly of claim 7, wherein the delivery port comprises the second port.

9. The portable insufflator assembly of claim 8, further comprising means for stepping down gas pressure from a carbon dioxide gas cannister to allow for, as an alternative to ambient air, delivery of carbon dioxide through a set of tubes through which the ambient air is delivered when the carbon dioxide cannister is disconnected from the medical insufflator assembly.

10. A medical insufflator assembly, comprising:
an access port fluidly coupled with and configured to provide access to ambient air;
a gas pump fluidly coupled with the access port;
a delivery port configured to deliver pressurized ambient air generated from the gas pump;
a filter assembly configured to filter ambient air received from the access port;
a pressure sensor configured to sense an internal gas pressure; and
a valve configured to open a venting port upon sensing a threshold pressure by the pressure sensor.

11. The medical insufflator assembly of claim 10, further comprising a battery configured to provide a portable energy source for delivery of electrical energy to the gas pump.

12. The medical insufflator assembly of claim 11, further comprising an alternating current converter to allow for use of alternating current for charging the battery or operating the medical insufflator assembly using an alternating current power source.

13. The medical insufflator assembly of claim 10, wherein the filter assembly is removable and replaceable.

14. The medical insufflator assembly of claim 10, further comprising an alarm configured to notify a user of a detection of a condition dangerous to a patient, the condition comprising at least one of a gas pressure and a gas flow rate exceeding a threshold.

15. A method for insufflating a patient during a surgical procedure, the method comprising the steps of:
drawing ambient air into an insufflation device, wherein the insufflation device comprises a portable insufflation device configured to operate using a direct current source;
pressurizing the ambient air within the insufflation device; and
delivering pressurized ambient air through an incision in a patient using the portable insufflation device.

16. The method of claim 15, wherein the direct current source comprises a battery.

17. The method of claim 15, further comprising:
filtering the ambient air through a filter assembly;
removing the filter assembly; and
replacing the filter assembly with a new filter assembly.

18. The method of claim 15, further comprising:
coupling a carbon dioxide gas cannister with the portable insufflation device; and
delivering carbon dioxide gas through an incision in a patient using the portable insufflation device.

19. The method of claim 18, further comprising:
sensing a gas pressure exceeding a threshold gas pressure; and
in response to sensing the gas pressure exceeding the threshold gas pressure, actuating a valve to prevent gas exceeding the threshold gas pressure from being delivered into the patient.

20. The method of claim 18, further comprising stepping down gas pressure from the carbon dioxide gas cannister from a first pressure at which the carbon dioxide cannister stores the carbon dioxide gas to a second, lower pressure to allow for, as an alternative to ambient air, delivery of carbon dioxide through a set of tubes through which the ambient air is delivered when the carbon dioxide cannister is disconnected from the portable insufflator device.
